(19) United States
(12) Patent Application Publication
(10) Pub. No.: US 2004/0115282 A1
(43) Pub. Date: Jun. 17, 2004

(54) Gelled laxative compositions

(76) Inventors: Dale A. Keiser, Wickenburg, AZ (US); Richard Harry Turner, Mercer Island, WA (US)

Correspondence Address: HALE AND DORR, LLP, 60 STATE STREET, BOSTON, MA, 02109

(21) Appl. No.: 10/681,791

(22) Filed: Oct. 8, 2003

Related U.S. Application Data
(60) Provisional application No. 60/417,328, filed on Oct. 9, 2002.

(51) Int. Cl. A61K 35/78; A61K 33/42; A61K 31/19

(52) U.S. Cl. 424/601; 424/738; 514/54; 514/57; 514/557

(57) ABSTRACT

The invention provides orally administered gelled formulations comprising a laxative which is active in the digestive tract.

This application claims the benefit of Provisional Patent Application No. 60/417,328 filed Oct. 9, 2002, and is herein incorporated by reference.

### FIELD OF INVENTION

This invention is in the field of gastroenterology, and relates to orally administered gelled formulations active in the digestive tract. More specifically, this invention relates to gelled laxative compositions that may be administered for preparing the colon for surgical or diagnostic procedures or childbirth.

### BACKGROUND

In order to carry out a number of medical procedures, such as colonoscopy, radiographic examination, and childbirth, and in preparation for patients undergoing bowel surgery, it is often critical that the colon be emptied as completely as possible.

A number of orally administered liquid pharmaceutical compositions have been developed for use as gastrointestinal washes for diagnostic purposes or for use as cathartic laxatives. Such preparations consist of aqueous solutions of polyethylene glycol and electrolytes such as sodium sulfate, sodium bicarbonate, sodium chloride and potassium chloride. These orally administered compositions are particularly useful in the rapid washing of the colon for diagnostic purposes. For example, when a powerful gastrointestinal wash is required, such preparations are generally administered in a quantity of about four liters, the composition being typically formulated according to the following: polyethylene glycol 59 g., sodium sulfate 5.68 g., sodium bicarbonate 1.69 g., sodium chloride 1.46 g., potassium chloride 0.745 g. and water to make up one liter and relatively thorough evacuation is often significantly improved over enema formulations, and generally without the problems often encountered with enema administrations.

The advantages of using these preparations over other orally administered preparations are a drastic reduction in wash time and the minimization of water and electrolyte losses. The advantages that these types of solutions provide are derived from two essential characteristics of the preparation, namely, its isoosmoticity with the physiological liquids, and the balance of the ion species in solution, so as to compensate the transport mechanisms which regulate gastrointestinal absorption. These characteristics result in substantial isotonicity between the preparation and the intracellular and extracellular fluids at the tissues of the digestive tube walls.

Commercially available product embodying these formulations typically utilize a polyethylene glycol formula serving as a non-absorbable osmotic agent with a mixture of electrolytes for replenishment, so that patients do not become dehydrated. Patients are required to ingest a significant amount of volume for purgation that may include one eight-ounce glass every ten minutes for a total of one gallon of fluid. Due to the fact that the volume is so high, use of this type of formulation is frequently associated with a tremendous amount of distention and significant amounts of nausea. Another serious drawback of these known preparations is their decidedly unpleasant, bitter, and noticeably saline taste which in the more sensitive patients can lead to vomiting thereby preventing ingestion.

In an attempt to avoid the problems associated with the high volume types of preparations, other investigators have utilized ingestible preparations that consist of aqueous solutions of phosphate salts. The aqueous phosphate salt solution produces a tremendous osmotic effect on the intra-luminal contents of the bowel, and therefore, evacuation of the bowel occurs with a tremendous increase in the influx of water and electrolytes into the colon. This has been developed for the express purpose of decreasing the volume required in colonic purgations. One such preparation manufactured by Fleet under the brand name Fleet PHOSPHO-SODA™ is manufactured according to the National Formulary monograph for Sodium Phosphates oral solution. This product, as described in the National Formulary (USP 23/NF18, p. 1430), contains dibasic sodium phosphate and monobasic sodium phosphate or phosphoric acid in water. Patients are typically required to take two 1.5 ounce dosages of this preparation, separated by a three to 12 hour interval for a total of three ounces, which is a significant reduction compared to the 128 ounces required by other high volume preparations. Gastroenterologists report excellent cleaning results with the concentrated aqueous phosphate solution.

However, the major shortcoming of such concentrated aqueous phosphate solution administration is that the aqueous solution is extremely unpalatable, so much so that the recommended dosage form is administered ice cold so as to minimize the objectionable saline taste. Often patients complain of severe nausea and vomiting, possibly secondary to the extremely salty taste of the preparation. Frequently, patients cannot even tolerate the ingestion of this preparation at the initial dose and often the second dose becomes even more problematic due to the unpalatable extremely salty taste, even when the taste is partially masked by the use of flavoring agents.

An additional shortcoming of such concentrated aqueous phosphate solution administration is the occurrence of side effects related to exposure of the intestinal lining to a rapid increase in phosphate salt concentration. Side effects include cramping, nausea, and vomiting.

Thus, while concentrated purgation solutions represent an improvement over other methods of inducing purgation, the unpalatable taste and the unpleasant side effects are serious shortcomings.

Other investigators have utilized capsules and tablets to contain and deliver dry formulations as a solution to the problem of unpalatable taste. (See U.S. Pat. No. 5,616,346 to Aronchick and U.S. Pat. No. 5,997,906 to Wood.) Gastroenterologists have reported reduced fecal cleansing and a problem with increased foam in the upper colon with these solid forms as compared to the aqueous forms, most likely a consequence of the use of binders and coatings in the formulations. The administration of these nonaqueous formulations typically requires that three tablets be taken at a time with eight ounces of clear liquid every 15 minutes for a total of 20 tablets. Then, 3-5 hours before the medical procedure, the process is repeated with another 20 tablets for a grand total of 40 tablets and 112 ounces of liquid. This regimen is quite demanding for a patient.

Thus, there is a need for an improved formulation which solves many of the problems associated with the high volume aqueous formulations, and is palatable without unpleasant side effects.

### SUMMARY

It has been discovered that combining a gel carrier with a pharmaceutically active component surprisingly results in a composition that temporarily masks the taste of the active component while preserving its efficacy. Additionally, this gelled composition provides for the slowed release of a high strength pharmaceutical component, such as a laxative, in the intestine so as to minimize side effects such as cramping and nausea that can occur with the otherwise rapid introduction of the pharmaceutical component.

These discoveries have been exploited to provide the present invention, which in one aspect includes a pharmaceutical composition comprising a gel component intermixed with a laxative component.

In one embodiment the gel component is selected from the group consisting of gelatin, pectin, carrageenen, agarose, agar, psyllium, cellulose, and agar-agar. In some embodiments, the laxative component is a cathartic laxative component. The laxative component is selected from the group consisting of bisacodyl, docusate, polyethylene glycol, sodium sulfate, magnesium sulfate, sodium bicarbonate, sodium chloride, potassium chloride, potassium sulfate, sodium phosphate, phosphoric acid, and magnesium citrate, and combinations thereof, in some embodiments of the invention.

In a particular embodiment, the laxative component comprises sodium phosphate, and in another particular embodiment, the sodium phosphate is present in the composition at a range of about 0.35 g to about 16 g per ml of liquid composition. As used herein, “liquid composition” refers to the gel and laxative components dissolved in a drinkable liquid, such as water. In specific embodiments, the sodium phosphate is present as about 0.25 to about 12 g monobasic sodium phosphate, and about 0.1 g to about 4.5 g dibasic sodium phosphate per 100 ml liquid composition.

In other embodiments, the laxative component comprises magnesium citrate or polyethylene glycol. In particular embodiments, the magnesium citrate is present in the composition at a range of about 1 g to about 11 g per ml liquid composition and the PEG is present at from about 1 g to about 30 g per 100 ml liquid composition. In some embodiments the gel component and the laxative component are in powdered form. In further embodiments, the composition also comprises at least one component selected from the group consisting of pharmaceutically compatible flavors, dyes, fragrances, stabilizers, and preservatives.

The invention also provides a kit for preparing a gelled laxative formulation. The kit comprises a gel, a laxative, and instructions on how to prepare and use the formulation. In one embodiment, the gel component and the laxative component are provided in powdered form.

In another aspect, the invention is directed to a method of cleansing the colon and bowels of a patient by orally providing a therapeutically effective amount of the pharmaceutical composition of the therapeutic composition to a patient.

### DETAILED DESCRIPTION

The present invention described here provides a gelled pharmaceutical laxative composition having a pleasant odor and taste and having the effect of reduced negative side effects compared to many known laxative formulations. This composition may be a colonic laxative to treat constipation or a cathartic or purgative formulation useful for cleansing the bowels before examination, surgery, or childbirth. As used herein, the term “laxative” may also encompass purgatives.

One of the negative side effects of many laxative formulations is unpleasant taste. Unpleasant taste is a result of drenching the taste receptors in the patient's mouth with the high concentration of salts present in the purgative formulations. The formulations of the present invention temporarily retain the laxative component within a carrier gel so that the taste receptors in the mouth are minimally exposed to the offending formulation. As the patient ingests the gel formulation, the patient tastes only that minute percentage of active component that is exposed at the surface of the gel. Chewing prior to swallowing exposes more gel surface area, yet only a very small percentage of the total active saline component is exposed. The laxative or purgative formulation is released from the gel in the stomach of the patient and small intestine as the gel dissolves. With the strong saline taste minimized, the flavoring and sweetener added to the carrier become evident to the patient. The gel formulation has a pleasant taste, is presented in a colorful and familiar form, and is readily accepted by the patient.

In addition to providing greatly improved taste over known formulations, the compositions of the present invention provide the further advantage of reduced side effects such as cramps and nausea, often associated with the known purgative formulations over known formulations. These side effects are a result of the shock induced by rapid changes in osmotic strength in the intestine. The rapid change in osmotic strength is the result of the rapid introduction, to the intestine, of the active pharmaceutical components of the known formulations. The instant compositions do not change the pharmaceutically active laxative component of the known formulations, but instead, provide for the slowed release of those components in the intestine. The slowed change in osmotic strength provided by the described embodiments proportionally reduces the shock otherwise associated with a rapid change in osmotic strength.

The therapeutic composition of the invention comprises a gel component as a carrier and a laxative component.

The type of gel component is not limited to any particular form, as long as it is edible and will not reduce the laxative activity of the laxative component. Useful commercially obtainable gels include, but are not limited to, pectin, agar, agarose, agar-agar, Arabic gum, xanthum gum, tragacanth gum, karaya gum, ghatti gum, guar gum, gellan gum, locust bean gum, alginic acid or its salt (e.g., sodiumalginate), carrageenan, gelatin, dextrin, starches (corn starch, rice starch, wheat starch, potato starch, pueraria starch, tapioca starch, carboxymethyl starch, hydroxypropyl starch, hydroxyethyl starch, chemically cross-linked starch, alpha-starch), celluloses (hydroxypropylmethyl cellulose, carboxymethyl cellulose, methyl cellulose, methylethyl cellulose, hydroxypropyl cellulose, crystalline cellulose), polyvinyl alcohol, polyvinylpyrrolidone, polyethylene glycol (macrogol), or mannans can be used singly or in an appropriate combination.

Useful commercially obtainable laxative components include, but are not limited to bisacodyl, docusate, polyethylene glycol (PEG), psyllium, cellulose, senna, sodium chloride, potassium chloride, potassium sulfate, sodium phosphate, phosphoric acid, and magnesium citrate, which may be used alone or in combination.

The amount of gel component and laxative component in the composition will depend on the chemical nature of each, such as the ability of the components to dissolve in the liquid. The components are present in therapeutically effective amounts and up to their limit of solubility in the liquid. The liquid composition refers to the components dissolved in a liquid, such as water.

For example, if gelatin is used as the gel component, and PEG is used as the laxative, the final gelatin concentration can be about 2.2 g to 10 g per 100 ml liquid composition, and the final PEG (e.g., PEG 3350) concentration can be about 1 g to about 30 g per 100 ml liquid composition. If salts are to be added, e.g., sodium phosphate, magnesium phosphate, and potassium phosphate (such as in ULVL, Braintree Labs, Braintree, Mass.), the final concentrations may be from about 1 g to about 10 g Na₂SO₄, from about 1 g to about 10 g Mg₂SO₄, and from about 0.5 g to about 2 g K₂SO₄, per 100 ml liquid composition.

If agar is used as the gel component instead of gelatin in the same PEG/salt liquid composition, its final concentration may be from about 1.5 g to about 10 g per 100 ml liquid.

If gelatin is used as the gel component and PEG is used as the laxative component, the final gelatin concentration may be from about 2.2 g to about 10 g per 100 ml liquid composition, and the final PEG 3350 concentration may be from about 1 g to about 30 g per 100 ml liquid composition. If agar is used instead of gelatin in this example, its final concentration may be from about 1.5 g to about 10 g per 100 ml liquid composition.

If sulfate salts are used as the laxative component and gelatin is used as the gel component the concentrations of these constituents, per 100 ml liquid composition are from about 1 g to about 15 g Na₂SO₄, from about 1 g to about 15 g Mg₂SO₄, from about 0.5 g to about 4 g K₂SO₄, and from about 2.2 g to about 10 g gelatin. If agar is used in this example instead of gelatin, the salt concentrations remain the same, and the final agar concentration is from about 1.5 g to about 10 g per 100 ml liquid composition.

If magnesium citrate is used as the laxative component and gelatin is used as the gel component, the final magnesium citrate concentration may be about 1 g to about 11 g per 100 ml liquid composition, while the final gelatin concentration may be about 2.5 g to about 10 g per 100 ml liquid composition. If agar is used instead of gelatin in this example, the final agar concentration may be from about 1 g to about 10 g per 100 ml liquid composition, and the magnesium citrate concentration may be increased to from 1 g to about 25 g per 100 ml liquid composition.

If sodium phosphate is used as the laxative component and gelatin is used as the gel component, the final gelatin concentration may be about 2 g to about 10 g per 100 ml liquid composition, and the final sodium phosphate concentration may be about 0.35 g to about 16 g per 100 ml liquid composition. If PHOSPHO-SODA™ is used as the sodium phosphate, the final monobasic sodium phosphate concentration may be about 0.25 to about 25 g per 100 ml liquid composition, and the final dibasic phosphate concentration may be about 0.1 g to about 10 g per 100 ml liquid composition.

PEG can be added as an additional laxative to either the sodium phosphate-, PHOSPHO-SODA™-, or magnesium sulfate-containing compositions.

The compositions of the invention may also include flavorings, dyes, fragrances, stabilizers, sweeteners, and/or preservatives, all known in the art. For example, the composition can contain flavorings such as cherry, grape, tea, apple, lemon-lime flavoring, etc., which may be oil-based. Such flavorings are commercially available from, e.g., International Flavors and Fragrances, New York, N.Y.

The solution can also or alternatively contain sweetenings such as sugar, sucralose, acesulfameK, fructose, and/or aspartame, which are also commercially available, e.g., from Sigma Chemical Co., St. Louis, Mo. or from Nutrinova Inc., Somerset, N.J. Flavor enhancers such as, but not limited to, malic acid citric acid, and/or ascorbic acid can be added. These enhancers are available from, e.g., Sigma Chemical Co., St. Louis, Mo. The solution can also be colored to match the flavor, e.g., light brown for apple juice, dark brown for tea, purple for grape, etc. Useful colorings can be commercially obtained from, e.g., McCormick and Company, Inc., Hunt Valley, Md. Preservatives can be added to keep freshness. Some useful preservatives include, but are not limited to, parabens, benzoates, sorbates, and alcohols, commercially obtainable from, e.g., Sigma Chemical Co., St. Louis, Mo. The solution may be unclear (cloudy, a suspension, etc.) with additives for product effect to look like orange juice, iced tea, and other drinks. Other known additives can be used and the formula modified to optimize taste, odor, stability, solubility, acidity, color, etc.

One nonlimiting embodiment of the composition of the invention provides a gel with a consistency similar to that of the familiar JELL-O™ brand desert. However, the type or concentration of gelling agent may be adjusted to produce different degrees of firmness of the gel as well as to change other physical properties of the gel such as melting temperature and texture. The properties of the gel component may be selected to optimize the characteristics of an end formulation that may or may not include flavorings, sweeteners, fragrances, dyes, stabilizers, or preservatives, as described above.

For example, in one embodiment, the gel component further comprises a flavoring, a sweetener, a dye, and/or a preservative, and the laxative component comprises dibasic sodium phosphate and monobasic sodium phosphate according to the National Formulary monograph for Sodium Phosphates oral solution (USP 23/NF18, p. 1430). This embodiment Comprises a commercially available gelatin desert, for example, JELL-O™ brand desert mix, and a purgative, for example Fleet brand PHOSPHO-SODA™. The desert mix provides gelatin, flavoring, dye, sweetener, stabilizer, and preservative. The exemplary PHOSPHO-SODA™ provides the appropriate ratio of dibasic sodium phosphate and monobasic sodium phosphate (according to the National Formulary monograph for Sodium Phosphates oral solution (USP 23/NF18, p. 1430)) plus additional stabilizers, preservatives, and flavoring.

To prepare an eight-ounce serving of the composition of the invention, three ounces of the exemplary orange flavored JELL-O™ brand desert mix is combined with 130 ml of water. The solution is heated to near boiling while stirring until solids are dissolved. In a separate container, 65 ml of water are combined with 45 ml of Fleet brand PHOSPHO-SODA™, and the solution is heated to near boiling. The diluted purgative is combined slowly into the gelatin mixture while the mixture is stirred. The mixture is slowly cooled in an eight-ounce container to about 35° F. This mixture Provides an eight ounce serving of gel containing an effective 1.5 ounce purgative dose.

In another embodiment, the carrier component comprises gelatin, a flavoring, a sweetener, a dye, and a preservative, and laxative component is magnesium citrate. The gelatin in this example formulation is, for example, Gelatin, Type A, 25 Bloom, 50 mesh (from Great Lakes Gelatin, PO Box 917, Grayslake, Ill. 60030). The purgative is, for example, Long's Drug Co. brand Magnesium Citrate Oral Solution™. The Long's Magnesium Citrate Oral Solution™ provides flavoring, dye, sweetener, stabilizer, and preservative. To prepare this pharmaceutical composition, 10 fluid ounces of the magnesium citrate solution is transferred to a container, to which 8.75 g of gelatin is added on top of the solution. The solution is stirred until the gelatin is dispersed. When the gelatin has hydrated (about 15 min.), the solution is heated to near boiling while being stirred until all solids are dissolved. The mixture is slowly cooled to about 35° F. This example provides a 10-ounce serving of gel containing an effective dose of oral magnesium.

In another embodiment, the gelled carrier component comprises agar, e.g., Sigma brand Agar A-7002 Lot 71K0093), and the laxative component comprises magnesium citrate (e.g., Brite-Life Pasteurized Magnesium Citrate Oral Solution, Westburg Pharmacy, Richmond, Va.). The magnesium citrate oral solution provides flavoring, dye, sweetener, stabilizer, and preservative. The magnesium citrate solution can have a final concentration of 1 to 11 g per 100 ml.

To prepare this composition, two grams of agar is added to the 118 ml (2 oz.) of magnesium citrate solution. The agar is stirred to disperse it. The resulting mixture is heated to near boiling while being stirred until the solids are dissolved. The mixture is cooled slowly to about 35° F. This example provides an 2-ounce serving of gel.

In some embodiments, the gelled laxative composition is into a single mass. However, the gel alternately may be molded into any of several forms including various diameter balls, discs, strips, or squares. For example, a small ice cube tray may be used to mold several one-half inch cubes. A dose may comprise a number of smaller-than-bite-size gel shapes that are stirred into a glass of water and then drunk without chewing.

Each of the previously described embodiments is a gelled formulation that may be presented to the patient in a ready-to-use gel form. Alternatively, the formulation can be prepared and presented to the patient in forms other than a ready-to-use gel whereby the patient may prepare the gel form. Several methods of making and supplying the components of the gel formulation are possible.

For example, the gel and laxative components, along with any of the additives mentioned above may be supplied separately or mixed together with instructions for preparation and use can be supplied in a single package or kit. The patient prepares the composition by adding a liquid such as water or Gatorade to the dry components, then heating, mixing, and cooling the mixture. The dry components and liquid to be added thereto can also be supplied separately in a single package whereby the patient may prepare the gel by appropriately mixing, heating, and cooling the mixture. Alternatively, the composition may also be supplied to the patient in a liquid form such that when the liquid is cooled, it forms a gel.

The laxative compositions of the invention mask the taste of the laxative/purgative component as long as the composition is in its gelled form. The gelled composition will melt, depending on the specific composition, at elevated temperature. In the case of compositions that will melt at a temperature less than the temperature of a patient's mouth, the method of taking the compositions become important.

For example, the gelled composition may be chilled to a temperature in the range of 30° to 37° F. The composition will remain gelled for a longer time if it is colder when taken by the patient.

The gelled composition may be taken in small portions, such as portions less than one teaspoon, so that each portion may be swallowed after minimal chewing. In some cases, the portion would be swallowed with no chewing.

The patient may drink a small amount of cold liquid before, and/or with, and/or after each portion of the composition. This method cools the patients mouth resulting in less heat transfer to the gelled composition and hence, less melting of the gel. This method also rinses away any of the unpleasant tasting component that may have escaped the gel, and encourages increased fluid intake as generally prescribed for the bowel cleansing process.

The foregoing descriptions are illustrative of several embodiments. The descriptions are not intended to limit the invention to the specific formulations shown and described, but instead it will be appreciated that adaptations and modifications will become apparent from the present disclosure and are intended to be within the scope of the claims.

We claim:

1. A pharmaceutical composition comprising:
a) a gel component, intermixed with
b) a laxative component.

2. The composition according to claim 1, wherein the gel component is selected from the group consisting of gelatin, pectin, carrageenen, agarose, agar, psyllium, cellulose, and agar-agar

3. The composition of claim 1, wherein the laxative component is a cathartic laxative component.

4. The composition according to claim 1, wherein the laxative component is selected from the group consisting of bisacodyl, docusate, polyethylene glycol, sodium sulfate, sodium bicarbonate, sodium chloride, potassium chloride, potassium sulfate, magnesium sulfate, sodium phosphate, phosphoric acid, and magnesium citrate, and combinations thereof.

5. The composition according to claim 4, wherein the laxative component comprises sodium phosphate.

6. The composition according to claim 5, wherein the sodium phosphate is present in the composition at a range of about 0.35 g to about 16 g per ml of liquid composition.

7. The composition according to claim 5, wherein the sodium phosphate is present as about 0.25 to about 12 g monobasic sodium phosphate, and about 0.1 g to about 4.5 g dibasic sodium phosphate per 100 ml liquid composition.

8. The composition according to claim 4, wherein the laxative component comprises magnesium citrate.

9. The composition according to claim 8, wherein the magnesium citrate is present in the composition at a range of about 1 g to about 11 g per ml liquid composition.

10. The composition of claim 4, wherein the laxative formulation comprises polyethylene glycol (PEG).

11. The composition of claim 10, wherein the PEG is present at from about 1 g to about 30 g per 100 ml liquid solution.

12. The composition according to claim 1, wherein the gel component and the laxative component are in powdered form.

13. The composition according to claim 1, further comprises at least one component selected from the group consisting of pharmaceutically compatible flavors, dyes, fragrances, stabilizers, and preservatives.

14. A kit for preparing a gelled laxative formulation, comprising:
(a) a gel,
(b) a laxative, and
(c) instructions on how to prepare and use the formulation.

15. The kit of claim 14, wherein the gel component and the laxative component are provided in powdered form.

16. A method of cleansing the colon of a patient by orally providing a therapeutically effective amount of the pharmaceutical composition of claim 1 to a patient.
